(19) United States
(12) Patent Application Publication
(10) Pub. No.: US 2022/0000993 A1
(43) Pub. Date: Jan. 6, 2022

(54) DRUG FOR USE AGAINST THE NOVEL CORONAVIRUS DISEASE, COVID-19

(71) Applicant: Torvald Ranta Foretagsjuridik AB, Stocksund (SE)

(72) Inventor: Alexandr Vladimirovich Khanykov, Moskow (RU)

(21) Appl. No.: 17/367,962

(22) Filed: Jul. 6, 2021

Related U.S. Application Data
(60) Provisional application No. 63/048,391, filed on Jul. 6, 2020.

(51) Int. Cl. A61K 38/57; A61K 9/00; A61K 31/675; A61K 31/573; A61K 31/4439

(52) U.S. Cl. CPC A61K 38/57; A61K 9/0075; A61K 31/4439; A61K 31/573; A61K 31/675

(57) ABSTRACT

The invention concerns a medicinal drug effective for the treatment of COVID-19 disease caused by the SARS-Cov-2 virus and preventing the development of its dangerous complications associated with pulmonary edema. For this purpose, a drug is proposed comprising aprotinin as a pharmaceutically active component in the form of a dry powder aerosol with a particle size of less than 1.0 μm. Such a drug provides the possibility of targeted delivery of aprotinin to the lower respiratory tract and alveoli, which makes it possible to influence both the etiology (due to the antiviral effect of aprotinin) and the pathogenesis of COVID-19 disease (due to the anti-inflammatory effect of aprotinin) directly in the focus of the pathologic pathway.

### CROSS-REFERENCE TO RELATED PATENT APPLICATIONS

This application claims the benefit of U.S. Provisional Patent Application No. 63/048,391, filed Jul. 6, 2020.

### FIELD OF THE INVENTION

The use aprotinin in a form of a dry aerosol is recommended for the prevention and treatment of the novel coronavirus disease, COVID-19.

### BACKGROUND OF THE INVENTION

Currently, the search for drugs for preventing the spread of and treating patients with the coronavirus disease COVID-19 is of great practical importance due to the high morbidity and mortality from this disease during a pandemic. However, it is very difficult to find an effective drug due to the fact that the virus is new, the mechanism of its effects on humans is not fully understood, and the lack of accurate information on the pathogenesis of the infection caused by it.

Over the months of the coronavirus pandemic, we have gathered sufficient information to claim that the risk of mortality is mostly determined by the presence of two events: a cytokine storm and DIC (Disseminated Intravascular Coagulation). Proteases are enzymes that initiate protein degradation and play a key role in these processes.

Moreover, it has been proven that proteolysis plays a significant role in the development of the most dangerous viral infections, in particular, influenza. It has also been acknowledged that the virus COVID-19 uses the protease TMPRSS2 to enter cells by increasing the activity of blood proteases and stimulating the formation of kinins (bradykinin, kallikrein and other cytokines) in tissues. Microcirculatory disorders and the increase of vascular permeability leads to an accumulation of transudate in the interstitium, partially in the alveoles, resulting in edema of the alveolar-capillary membrane exudative and inflammation. These phenomena are the biochemical bases of cytokine storm and DIC which are the main causes of mortality in coronavirus pneumonia.

Because of the absence of etiotropic therapy, symptomatic therapy is usually prescribed for the treatment of COVID-19 such as antipyretics for the fever, analgesics for severe chest, throat, or muscle pain, and oxygen therapy. Drugs used to treat other infectious diseases, such as malaria, are currently undergoing clinical trials in patients with coronavirus.

Proposals include anticoagulant heparin that can block S-protein on the surface of coronavirus, such as the anti-oxidant and anti-inflammatory medicine N-acetylcysteine that can split this protein, and dexamethasone; famotidine, the active substance for heartburn, which is sold under the name Pepcid. The last one, as reported by Sciencemag.org, connects to the key enzyme responsible for the development of the acute respiratory syndrome coronavirus 2 (SARS-CoV-2), and can have a therapeutic effect.

The use of antiviral drugs with alpha- and/or gamma interferon have become widespread. A common disadvantage of these drugs is their lack of efficiency, especially in severe clinical cases, related to the absence of the combined impact on key pathogenesis stages of the disease.

Clinical trials of an antiviral drug, Remdesivir, have been extremely controversial and the American Food and Drug Administration (FDA) has not yet approved it for June 2020. It is an antiviral drug that inhibits RNA-directed RNA polymerases, and thus prevents viral replication in infected cells. However, side effects occur when using this medication in therapeutic concentration with parenteral administration. In addition, it does not prevent the “cytokine storm” and DIC, which are the main causes of ground-glass opacity.

Unfortunately, despite intensive research and development started around the world since the discovery of the SARS-Cov-2 virus, at the moment, an effective drug for the treatment of the coronavirus disease COVID-19, and most importantly, the prevention of the development of severe forms of the disease, is still not found.

### SUMMARY OF THE INVENTION

The most important purpose of COVID-19 therapy is to prevent the development of dangerous complications associated with pulmonary edema. Pulmonary edema caused by COVID-19 begins in the distal lungs when they fill with exudate due to cytokine storm and disseminated intravascular coagulation. Such edema is fundamentally different from the edema that occurs with pneumonia caused by other pathogens, since it is associated with an increase in capillary permeability due to the massive penetration of kinins (cytokines).

Fortunately, not every person has the virus causes processes that lead to such severe consequences, but in many patients, pulmonary edema occurs abruptly, uncontrollably and leads to death. At the same time, the leading link in the pathogenesis of such a development of the disease is not so much damage caused directly by the SARS-Cov-2 virus, but rather an overreaction of the organism.

It is well known that aprotinin has been used as an anti-inflammatory agent for more than 50 years, and is also used as a means of preventing blood loss during surgery. The mechanism for stopping bleeding with aprotinin is associated with strengthening the walls of damaged capillaries (traumatized during surgery) and preventing an increase in their permeability.

It is this effect of aprotinin that is primarily necessary to strengthen the capillaries of the alveoli in the first phases of the development of COVID-19.

Recent studies have also convincingly proved the antiviral efficacy of aprotinin associated with inhibition of the protease necessary for the replication of respiratory viruses, which was shown primarily for the example of the influenza virus and other viruses that cause acute respiratory viral infections (ARVI).

This ability of aprotinin to provide a binary—anti-inflammatory and antiviral—effect determines the potential effectiveness of aprotinin in the treatment of COVID-19 disease, aimed, among other things, at preventing the development of dangerous complications of this disease associated with pulmonary edema.

However, the available drugs aimed at delivering aprotinin to the respiratory tract were developed exclusively for the treatment of diseases (influenza, ARVI), in the pathogenesis of which the virus multiplication in the upper respiratory tract is of prime importance.

The formulation of various agents for the treatment of respiratory diseases (such as Hexoral, nasal sprays, water-oil solution of aprotinin (Aerus)) is designed in such a way so that aerosol particles (drops) containing the active substance settle in the upper respiratory tract.

At the same time, as indicated above, it is ineffective in conditions of COVID-19 disease, since the drug must be delivered to the lower respiratory tract and alveoli.

The objective of the present invention is to develop a drug (medicinal drug, medicine, pharmaceutical composition) effective for treating COVID-19 disease caused by the SARS-Cov-2 virus and preventing the development of dangerous complications associated with pulmonary edema.

The solution to this problem is carried out by developing a drug that is aprotinin in a form of a dry powder aerosol with a particle size of less (fewer) than 1.0 microns (μm).

In some embodiments, the drug further comprises at least one pharmaceutically acceptable additive.

In some particular embodiments of the invention, the pharmaceutically acceptable additive is a carrier and/or excipient.

In some particular embodiments, the pharmaceutically acceptable additive is lactose, mannitol, trehalose, sucrose, sorbitol, xylitol, and/or glucose. In some particular embodiments, the pharmaceutically acceptable additive is lactose.

In some particular embodiments of the invention, the drug is for administration using a dry powder inhaler.

In some particular embodiments of the invention, a dry powder of aprotinin with a particle size of less than 1.0 μm is placed in a gelatin capsule intended for use in dry powder inhalers.

In some embodiments, the drug comprises at least one additional pharmaceutically active ingredient.

In some particular embodiments of the invention, the additional pharmaceutically active component is selected from antibiotics or other antimicrobial substances, hormonal agents, potentiators.

In some particular embodiments of the invention, the additional pharmaceutically active ingredient is selected from omeprazole, dexamethasone and/or Remdesivir.

The solution to this problem is also achieved by implementing a method for treating COVID-19 disease caused by the SARS-Cov-2 virus and preventing the development of dangerous complications associated with pulmonary edema, including administering to the patient a drug containing aprotinin as an active component in a form of a dry aerosol with particle size less than 1.0 microns.

According to the invention, the drug is administered in a therapeutically effective amount.

In some embodiments of the invention, treatment is initiated at the earliest stages of infection with the SARS-Cov-2 virus.

In some embodiments of the invention, the treatment is carried out for a period of time sufficient for the elimination of the virus from the respiratory tract, as determined by testing (in particular, a test using PCR) for the presence of the SARS-Cov-2 virus.

The duration of treatment depends on the severity of the condition and can range from several days to several weeks. In some embodiments of the invention, the drug is administered from 5 to 14 days, in some particular embodiments from 5 to 7 days.

In some embodiments, the drug is administered using a dry powder inhaler.

In some embodiments of the invention, a single dose of a drug administered using a dry powder inhaler contains at least 85 KIU (Kallikrein Inhibitor Unit) of aprotinin.

In some embodiments of the invention, a single dose of a drug administered using a dry powder inhaler contains no more than 200 KIU of aprotinin.

In some embodiments of the invention, a single dose of a drug administered by a dry powder inhaler contains 85 to 150 KIU of aprotinin, in particular from 85 to 110 KIU of aprotinin.

In some embodiments, the drug is administered at least 5 times a day.

In some embodiments, the drug is administered 1 to 6 times a day, in particular 1 to 5 times a day.

In some private embodiments of the invention, the drug is administered every 2-3 hours.

The solution to this problem is also achieved with the use of a drug containing aprotinin as an active component in a form of a dry aerosol with a particle size of less than 1.0 microns, for the treatment of COVID-19 disease caused by the SARS-Cov-2 virus and prevention of the development of dangerous complications associated with pulmonary edema.

The use of a drug containing aprotinin as an active component in a form of a dry powder aerosol with a particle size of less than 1.0 μm provides the possibility of targeted delivery of aprotinin to the lower respiratory tract and alveoli, which allows it to act on both etiology (due to the antiviral effect of aprotinin) and on the pathogenesis of the disease COVID-19 (due to the anti-inflammatory effect of aprotinin) directly in the focus of the development of the pathological process.

The use of aprotinin in a form of a dry powder aerosol does not require the inclusion of additives in the preparation, for example, to ensure its stability, etc. (for example, those that ensure the stability of aprotinin in solution), except for the addition, if necessary, of an inert carrier or excipient, providing the necessary volume and preventing the adhesion of the particles of the preparation for its spraying in a form of a dry aerosol. Aprotinin in this form remains stable for a long time under normal conditions. The absence of additives excludes the possibility of developing hypersensitivity reactions to them.

The use of aprotinin in a form of a dry powder aerosol makes it possible to use dry powder inhalers for its administration, which require minimal patient coordination between breathing and actuation of the drug delivery device, which makes it easy to ensure the accuracy of aprotinin dosage to the foci of the development of the pathological process.

The use of aprotinin in a form of a dry powder aerosol allows, if necessary, to include additional pharmaceutically active substances in the composition of the drug, also in a form of dry fine powders, without fear of their interaction in the composition of the drug, which is impossible in a wet dosage form.

### TERMS AND DEFINITIONS

The following terms and definitions apply in this document unless otherwise stated explicitly. References to techniques used in describing the present invention refer to well known techniques, including modifying these techniques and replacing them with equivalent techniques known to those skilled in the art.

In the specification of this invention, the terms “include”, “comprise”, “contain” (“including”, “comprising”, “containing”) are interpreted as meaning “includes, among other things”. These terms are not intended to be construed as “consists only of”.

Aprotinin is a low-molecular natural polypeptide (58 amino acids) that is extracted from cattle lungs. It is usually used as an anti-inflammatory agent in the treatment of inflammation of the liver, kidneys, and other diseases (brand names include: Gordoks, Kontrikal, Trasilol, Aprotex, Veronarkap and others) in IV solution. The antiviral properties of aprotinin are also known. An aerosol of the aprotinin solution in the inhalation mixture “Aerus” with a particle size of 5.0-150 (50 on average) pm was proposed by the author of the present invention as an antiviral agent for the treatment of influenza (EP 2594283; RU 2425691).

By “dry powder inhaler” (DPI), as used herein, is meant any inhalation device designed to deliver a drug to the lungs in a form of a dry powder. In the present invention, there are no restrictions related to the design of the inhaler and the method of dosage of the drug. The main requirement for inhalers is the ability to deliver dry aerosols with a particle size of less than 1.0 microns to the lungs.

By “therapeutically effective amount (therapeutic dose)” is meant the amount of drug administered to a patient at which the expected therapeutic effect is most likely to occur. The exact amount required may vary from subject to subject, depending on numerous factors, such as the severity of the disease, age, body weight, general condition of the body, combination treatment with other drugs, etc. Administration of the drug according to the invention to a subject in need of treatment and/or prevention of a disease or condition, is carried out in a dose sufficient to achieve a therapeutic effect. When carrying out treatment and/or prophylaxis, the administration can be carried out both once and several times a day, more often in a form of a course administration for a time sufficient to achieve a therapeutic effect (from several days to a week, several weeks and up to months), while courses of drug administration can be repeated. In particular, in moderate forms of the disease, the single dose, frequency and/or duration of administration of the drug according to the invention can be increased.

The term “subject” means any mammal that is susceptible to infection with the SARS-Cov-2 virus, preferably the subject is a human.

The term “(inert) excipient/carrier” as used herein refers to a safe, non-toxic pharmaceutically acceptable compound that is used to prepare a pharmaceutical composition (medicinal drug) with a fine particles (micronized) form of aprotinin.

Unless otherwise specified, technical and scientific terms in this application have the standard meanings generally accepted in the scientific and medical literature.

### DETAILED DESCRIPTION

It is known that the development of respiratory diseases caused by the vast majority of viruses begins in the upper respiratory tract. Therefore, the drugs used are aimed at suppressing the virus precisely in these parts of the respiratory system.

The difference between the SARS-Cov-2 coronavirus, which causes COVID-19, from the vast majority of viruses that cause respiratory diseases, is that after entering the respiratory tract, the epithelial cells of the distal lower respiratory tract become the main targets of the virus. Diffuse damage to alveolocytes and capillaries determines rapid interstitial and alveolar edema and severe pathology.

Therefore, drugs widely and successfully used for the treatment of respiratory viral diseases, intended for administration into the upper respiratory tract, are ineffective in the treatment of COVID-19 disease caused by the SARS-Cov-2 coronavirus.

The ability of aprotinin to have a dual (anti-inflammatory and antiviral) effect on the development of COVID-19 disease determines the potential effectiveness of aprotinin in the treatment of this disease, including preventing the development of its dangerous complications associated with pulmonary edema.

Even the minimum dosage of aprotinin has been shown to prevent the replication of influenza, coronavirus, and other viruses that cause respiratory infections. Based upon the results of the medical cases of over 300 patients with influenza, aprotinin has proven to be highly efficient even if it is used on the second or third day of the disease, when the virus has already multiplied significantly in the lungs. No resistant forms of the virus to this drug were observed.

To penetrate the cell, the coronavirus COVID-19 uses protein protease TMPRSS2 (Markus Hoffmann, Hannah Kleine-Weber, Simon Schroeder). SARS-CoV-2 cell entry depends on ACE2 and TMPRSS2 and is blocked by a clinically proven Protease Inhibitor. In the structure of the S-protein from the SARS-CoV-2 virus a loop was discovered consisting of 24 amino acids that does not exist in other human coronaviruses. Coronaviruses use this S-protein on its surface as a spike to attach itself to the receptors of attacked cells. Researchers assume that this flexible loop on the surface of the S-protein is available for proteolysis, thereby providing a high possibility for the virus to penetrate an attacked cell.

So basically, there is a mechanism of proteolytic activation of SARS-Cov-2 which is almost identical to that of the influenza virus, which means that the role of protease inhibitors is crucial for slowing the infectious process. The authors note that the S-protein of the new coronavirus connects to cell receptors almost 10 times tighter than the protein of the SARS-CoV-1 virus. Moreover, the authors explain the specific pathogenicity of the new virus by the fact that S-protein can be activated by furin protease. This protease is found not only in the lungs, but in all tissues of the body, and is the cause of systemic inflammation in the body.

aprotinin blocks exactly this type of protease, which significantly inhibits the penetration of the virus into the epithelial cells of the respiratory tract, thus interrupting the infection process at an early stage. At the same time, the ability of aprotinin to inhibit fibrinolysis and to inhibit the formation of kinins (bradykinin, kallikrein and other cytokines) in tissues, which cause a microcirculation disorder and an increase in vascular permeability, is implemented simultaneously.

As a result, aprotinin serves as an inhibitor of almost all plasma procoagulant and anticoagulant units, which are in a state of “explosion” during DIC. By inhibiting numerous mediators, aprotinin attenuates the inflammatory response, reduces fibrinolysis and the formation of thrombin, and significantly reduces spontaneous hemorrhagic events. (Berling R, Borgström A, Ohlsson K. Peritoneallavage with aprotinin in patients with severe acute pancreatitis. Effects on plasma and perito-neal levels of trypsin and leukocyte proteases and their major inhibitors; Khan M. M, Gikakis N, Miyamoto S, Rao A. K, Cooper S L, Edmunds L. H. Jr, Col-man R. W aprotinin inhibits thrombin formation and monocyte tissue factor insimulated cardiopulmonary bypass.)

This suggests that aprotinin significantly limits the systemic inflammatory response, which leads to the interconnected activation of hemostatic systems, fibrinolysis, and the activation of the cellular and humoral response. It is able to inhibit the release of anti-inflammatory cytokines and supports glycoprotein homeostasis. The mechanisms described above, acting together, prevent the onset of the phlogistic cascade and its inevitable consequences in the form of a persistent narrowing of the bronchi, a thickening of the bronchial wall and blood vessels, vascular leakage, etc., which manifested itself in computed tomography of the lungs in the form of “frosted glass”.

However, the use of aprotinin in the form of a water-in-oil aerosol for the treatment of COVID-19 is not effective enough, since the drug can not be delivered to the small bronchioles and alveoli due to its particle size. (In the case of influenza, this circumstance does not have such significance as mainly small and medium bronchi are affected).

The present invention is directed to the development of a dosage form of aprotinin for delivery directly to the lower respiratory tract and alveoli. And also, it is directed on the development of a method for treating COVID-19 disease by delivering aprotinin to the lower respiratory tract and alveoli.

It is known that for the delivery of a pharmaceutically active substance to the alveoli through the respiratory tract, the particles of the substance must be presented in a highly dispersed form. At the same time, it is known that aerosols with a high degree of dispersion are more stable: aerosol particles can remain in suspension for a long time, settle more slowly, and freely penetrate into the tracheobronchial tree. So, aerosols with particles of 0.5-1 microns practically do not settle on the mucous membrane of the upper respiratory tract, penetrating into the lower respiratory tract. Particles ranging in size from 2 to 5 microns predominantly settle on the walls of the alveoli and bronchioles. Medium-dispersed particles ranging in size from 5 to 25 microns are deposited in the bronchi of the II-I order, large bronchi, trachea. At the same time, it has been proven that particles larger than 10 microns do not penetrate deeper than the trachea.

However, as the results of the studies have shown, the currently existing aprotinin preparations do not allow them to be used to deliver the active substance to the lower respiratory tract and alveoli. This is due to the fact that water-oil aerosols of aprotinin (e.g. Aerus) do not allow the creation of stable highly dispersed particles, since aerosol droplets, due to surface tension forces, quickly agglomerate into larger ones and, as a result, settle in the upper respiratory tract without getting into the lower Airways. Intravenous administration of aprotinin also does not provide sufficient delivery of aprotinin to the lower respiratory tract.

Thus, despite the expected high efficacy of aprotinin in the treatment of COVID-19 disease, and the prevention of the development of dangerous complications associated with pulmonary edema, which are of great practical importance due to the high morbidity and mortality from this disease during a pandemic, there is currently no such a dosage form of aprotinin, which would ensure its effective delivery to the lower respiratory tract and alveoli.

Analysis of the amino acid composition of aprotinin showed that due to the predominance of polar positively charged amino acids, the aprotinin molecule is weakly positively charged, and thus, in a dry aerosol, equally charged particles will not agglomerate, since equally charged particles are repelled from each other under the action of electrostatic forces.

It was decided to conduct a study of the possibility of using aprotinin in a form of a dry lyophilized fine powder for the introduction into the lower respiratory tract. Such a powder can be obtained, for example, by mechanical grinding or mealing into a powder, or using spray drying or spray freeze drying.

For the study, aprotinin lyophilisate was used, ground into powder to particles no more than 1.0 μm in size. In preliminary experiments on animals (rats of the Wistar line), the fact was confirmed that when aprotinin is administered in this form, about 60% of aprotinin particles enter the lower respiratory tract. This allows us to expect that with the therapeutic use of dry aprotinin inhalations in patients, at least half of the inhaled dose will enter the lower respiratory tract.

For the administration of dry aerosol of aprotinin to patients, dry powder inhalers can be used to deliver the drug to the lungs in a form of a dry powder. Dry powder inhalers are portable devices that require minimal patient coordination between breathing and actuation of the powder medication delivery device. At present, dry powder inhalers of a wide variety of designs are known, which allow the administration of dry aerosols with varying degrees of powder dispersion. The main requirement for inhalers in the framework of the present invention is the ability to deliver a dry aerosol with a particle size of less than 1.0 μm to the lower respiratory tract.

The use of inhalations of a dry aerosol of aprotinin is possible both in mobile and stationary versions, depending on the patient's condition.

In some embodiments, these may be passively actuated devices that are actuated by the patient's breath. One of the variants of such a device provides for loading a gelatin capsule with a dose of aprotinin powder. When the device is triggered, the capsule is punctured and the impeller rotates the powder released from the capsule by the patient's inhalation force. Foil blisters, for example, can also be used instead of capsules.

In some other embodiments of the invention, actively-actuated dry powder inhalers can be used, the design of which provides an internal energy source for aerosol spraying of the powder, which eliminates the dependence of the volume of the administered dose of the drug on the patient's inhalation flow rate. In such devices, aprotinin powder is atomized by vibration, gas discharge, or impeller driven by a battery-powered motor, which can be activated by very small patient movements. Such inhalers are very convenient for use by fragile patients.

In some embodiments of the invention, the dry powder inhaler also has visual and/or audible feedback control mechanisms for controlling drug loading into the inhaler and inspiratory maneuver, informing the patient that the dose has been delivered correctly.

In the steady-state version, providing for use, for example, in medical clinics, compressor inhalers can be used, consisting of a compressor and a nebulizer (spray chamber). Moreover, spraying can be carried out both with air and helium-oxygen mixtures heated to a temperature of 40-60° C., to normalize the bronchopulmonary system (RU 2011121520; RU 2291718). Combining the use of aprotinin and the helium-oxygen mixture provides a synergistic effect and it is promising for the treatment of patients with coronavirus.

In addition to the obvious advantages of the invention over the use of aprotinin in water-oil mixtures, the use of dry powder inhalers for administration is another advantage of the invention. Although wet inhalers have long been used to administer aprotinin in oil/water mixtures, their use requires skill in order to follow the necessary sequential steps to use these devices. The improper completion of one or more steps when using such inhalers can significantly reduce the delivery of the administered drug and, therefore, its effectiveness. Numerous studies have shown that many patients do not use their inhalers correctly, and patients often do not know that they are using their inhalers incorrectly.

It is known that in non-optimized powder preparations, adhesion between particles (mainly associated with the van der Waals force) is possible, which causes aggregation of particles with the repeated formation of large particles. To prevent adhesion, the dry powder formulation of aprotinin may additionally include neutral carriers to provide better flowability and more accurate dose formulation of the formulation for inhalation. In some embodiments, such a carrier can be, but is not limited to, lactose, mannitol, trehalose, sucrose, sorbitol, xylitol, and/or glucose. To prevent adhesion, the surface of the carrier is usually spherical or irregularly shaped with ridges. If necessary, such pharmaceutically neutral excipients can be used as excipients.

In earlier studies of the authors of the invention (RU 2425691), it was proved that the therapeutic effect of aprotinin is found in the case of five inhalations during the day with doses of 85 KIU (i.e., the total daily dose is about 500 KIU). Studies have shown the absolute safety of using such dosages of aprotinin by inhalation, despite the fact that intravenous injections even for children are allowed in an amount of 2·106 KIU per day. Thus, long-term practice has confirmed the safety of aprotinin even when administered intravenously in doses 4000 times higher than those proposed by the authors for inhalation.

According to the invention, dry inhalation powder of aprotinin can be administered in single doses of 85-200 KIU, in private versions of the invention—85-150 KIU, in other private versions—85-110 KIU, which can be administered several times throughout the day as aerosol administrations in the nose or mouth every 2-3 hours, the estimated daily dose is at least 300-500 KIU/day per patient.

Nevertheless, to achieve an effective therapeutic effect, it is necessary to ensure the optimal accessibility of aprotinin into the bronchopulmonary system. Within the framework of the present invention, it was decided to abandon the use of the water-in-oil solution of aprotinin and to introduce it as a dry lyophilized powder with a particle diameter of less than 1.0 micron. This modification has significantly increased the active surface of the drug and now allows it to penetrate into the distal bronchi and alveoli to prevent severe scenarios in the development of the disease. It is worth noting that when inhaled by pulmonary administration (in contrast to the intravenous use of aprotinin, in which it is used in surgical operations to reduce blood loss), it does not enter the bloodstream, but instead decomposes in the lungs for 3-4 hours where it is utilized by enzyme and cell structures.

In addition, the use of aprotinin in dry form, and not in a form of a solution, protects aprotinin in the composition of the drug from hydrolytic reactions to which peptides and proteins are prone, such as deamidation, proteolysis and racemization, which contributes to its long-term storage without the addition of additional stabilizers and preservatives. This, in turn, simplifies and cheapens the production of the drug and excludes the possibility of hypersensitivity reactions to such excipients.

The aerosol administration of lyophilized aprotinin powder in COVID-19 patients will facilitate a reduction in mortality, and for mild versions of the disease, will significantly improve the patient's subjective perception and maintain the patient's working capacity during treatment. In addition, the effectiveness of aprotinin as a protease inhibitor has demonstrated that anti-SARS-CoV-2 activity can be further enhanced by therapeutic concentrations of Omeprazole (by 2.7 times) or Remdesivir (by 10 times). This confirms the possibility of using a complex preparation based on aprotinin.

aprotinin has never been proposed as a “pharmacological platform” for the pathogenetic therapy of COVID-19, and even less as a preventative therapy. Moreover, the inhaler drug delivery method for the treatment of COVID-19 has never previously been offered. The use of a complex formula for these purposes, where dry lyophilizates of various drugs form a single formulation is also unknown.

The use of aprotinin is especially promising with regard to its safety for the patient. This can be observed in more than 50 years of experience of using aprotinin worldwide for various inflammatory diseases and surgeries. aprotinin aerosols and Aerus sprays, in particular, are permitted for the treatment of patients. In this case, the inhaler usage of aprotinin is carried out in dosages a thousand times lower than the amount that enters the body when administered intravenously.

Using aerosol is carried out in a mobile or stationary version depending on the condition of the patient.

The mobile version is used for preventive measures or in the milder stages of the disease. It is based on a dry powder inhaler (DPI). aprotinin lyophilisate is thus placed in gelatin capsules or foil blisters that are embedded in a single-dose device, or contained in a reservoir with a medicinal substance, from which doses are measured using a multi-dose inhaler. To enhance the effect, two more capsule sets are introduced, in addition to a foil blister with pure aprotinin, where aprotinin is mixed with drugs that increase the effectiveness or breadth of its effect

In particular, aprotinin can be mixed with an antibiotic to achieve antimicrobial action, which has never been used in such drugs. In this way, this tool can be recommended as a “virtual mask” to increase the level of protection against the penetration of the virus into the lungs and used in areas that are at an increased risk of infection, such as hospitals.

So as to reduce the inflammatory component, the agent may contain hormonal preparations, such as dexamethasone, whereas it may contain Remdesivir, to enhance the direct antiviral effect of the drug.

Based on the foregoing, we can conclude that aprotinin is a unique drug agent that affects all three main pathogenetic events of COVID-19: proteolytic activation of the virus, cytokine storm, and microcapillary thrombosis. At the same time, aprotinin is a naturally occurring substance and it is used in minimal (homeopathic) doses, which prevents overdose and the development of side effects, such as an allergic reaction.

Although the invention has been described with reference to the disclosed embodiments, it should be apparent to those skilled in the art that the specific experiments described in detail are provided for the purpose of illustrating the present invention only and should not be construed as in any way limiting the scope of the invention. It should be understood that various modifications are possible without departing from the spirit of the present invention.

What is claimed is:

1. A medicinal drug for a treatment of COVID-19 disease and the prevention of its complications associated with pulmonary edema, including aprotinin as an active component in a form of a dry aerosol with a particle size of less than 1.0 μm.

2. The medicinal drug according to claim 1, further comprising a pharmaceutically acceptable carrier or excipient.

3. The medicinal drug according to claim 2, wherein the pharmaceutically acceptable carrier or excipient is selected from lactose, mannitol, trehalose, sucrose, sorbitol, xylitol or glucose.

4. The medicinal drug according to claim 1 for administration using a dry powder inhaler.

5. The medicinal drug according to claim 1, further comprising omeprazole, dexamethasone or Remdesivir.

6. A method of treating COVID-19 disease and preventing its complications associated with pulmonary edema, comprising: administering a medicinal drug containing aprotinin as an active component in the form of a dry aerosol with a particle size of less than 1.0 μm.

7. The method of claim 6, wherein the medicinal drug is administered using a dry powder inhaler.

8. The method of claim 7, wherein a single dose of the medicinal drug administered with the dry powder inhaler contains at least 85 KIU of aprotinin.

9. The method according to claim 8, in which one dose of the medicinal drug administered using the dry powder inhaler contains 85-200 KIU of aprotinin.

10. The method of claim 6, wherein the medicinal drug further comprises a pharmaceutically acceptable carrier or excipient.

11. The method of claim 10, wherein the pharmaceutically acceptable carrier or excipient is selected from lactose, mannitol, trehalose, sucrose, sorbitol, xylitol or glucose.

12. The method according to claim 6, wherein the medicinal product further comprises omeprazole dexamethasone or Remdesivir.
